(19) United States
(12) Patent Application Publication
(10) Pub. No.: US 2011/0275973 A1
(43) Pub. Date: Nov. 10, 2011

(54) HIV FILTRATION MACHINE AND METHOD OF FILTERING HIV USING THE MACHINE AND METHOD OF DETECTING HIV VIRUS DURING FILTRATION

(76) Inventors: Mohammad Saklain Rizvi, Baroda (IN); Mukulbhai Thakorbhai Amin, Baroda (IN); Kantibhai Ambalal Patel, Baroda (IN)

(21) Appl. No.: 12/737,899

(22) PCT Filed: Mar. 3, 2009
(86) PCT No.: PCT/IN2009/000140

§ 371 Date: Jul. 15, 2011

(30) Foreign Application Priority Data
Aug. 29, 2008 (IN) 1834/MUM/2008

(51) Int. Cl. A61M 1/34; B01D 21/30

(52) U.S. Cl. 604/5.02; 210/143

(57) ABSTRACT

A HIV filtration machine and method of filtration using the machine and method of detecting HIV, the said machine comprising an acrylic cylindrical tube divided into three portions i.e. needle portion (2), barrel portion (10), and tray (16), characterized in that the needle portion consists of three needle points (3, 4, 5), the said needle points are detachably connected with three blood carrying tubes (27, 28, 29) serially fixed into barrel portion, a valve (12) is provided at the end point of said blood carrying tubes, a piston (9) is detachably fixed above the plunger (22) and connected with a spring for creating movement of the plunger towards the tray, the said tray is one inch or less diameter in round shape and connected with steel ring of filter (14) and is connected with chip and micro camera, for data transmission to the remote computer or mobile device, the said filter having less than 1 micron to more than 8.5 micron.

### FIELD OF INVENTION

The present invention relates to a HIV/Aids filtration machine and method of filtering the blood such as to recover a HIV virus from the infected blood from the human body, which directly filters the HIV virus or HIV infected T cells, which are dangerous for human life. The device invented could be highly useful for sterile filtration of blood more particularly filtration of HIV virus from infected blood.

### BACKGROUND OF THE INVENTION

The present invention relates to a blood filtration machine and method of filtering the blood and method of detecting HIV virus in the body during filtration process for complete cure of which directly filters the HIV virus or HIV infected T cells which are dangerous for human life.

U.S. Pat. No. 4,466,888 teaches about blood collecting device, which collects blood having a collecting bag is clamped along its peripheral edges between the two parts of rigid shell.

U.S. Pat. No. 5,269,924 describes a blood collecting and filtering apparatus comprising a container with a removably fixed cover and a tightly closed rigid enclosure, said cover being provided with an adjustable device for connecting to a reduced pressure flexible, line and presenting a traversing opening and a disposable flexible filtration bag disposed in service position inside of said enclosure.

U.S. Pat. No. 7,220,236, provides a method for eliminating HIV in human blood and a device for treating AIDS with non-pharmacotherapy. The method includes (1) pumping the blood containing HIV virus into a soft thin plastic tube while adding air through a T-tube at a definite frequency so that the blood is evenly divided into very small blood droplets (2) introducing the small blood droplets into a screw-shaped quartz tube and exposing the quartz tube under a definite dosage of radiation to kill the HIV virus in the blood droplets and (3) collecting the treated blood in a storage bottle. Results show that after the HIV infected blood is divided into very small blood droplets and is irradiated with a definite dosage of ultraviolet (253.7 nm) for 90 seconds, more than 92% HIV loads in the blood is eliminated, while the lymphocyte (CD4+), erythrocyte, leucocyte and haemoglobin remain almost unchanged.

U.S. Pat. No. 7,226,429 discloses a method of reducing viral particles and lectin binding fragments thereof in the blood of an individual infected with a virus, comprising the steps of: a) obtaining blood from the individual; b) passing the blood through a porous hollow fiber membrane, wherein lectin molecules are immobilized within a porous exterior portion of the membrane, and wherein the lectin molecules bind to high mannose glycoproteins; c) collecting pass-through blood; and d) reinfusing the pass-through blood into the individual.

Whereas in the alleged invention describes about blood collecting for HIV filtration and filtering machine having a piston which creates push and pull movement causing of push and pull movement of plunger due to this pull push motion of plunger the blood will press toward the tray, which is connected via a filters steel ring, tray having microscopic camera for detection of HIV virus during filtration process. This gives strength to the filter for gentle and smooth filtration of blood for HIV filtration.

The HIV or HIV infected cells or other component of blood is to be passed and thrown across the filter by the pressure of plunger into the blood loaded barrel towards the tray. Therefore an alleged invention is novel and inventive over the cited documents.

### SUMMARY OF THE INVENTION

Present invention describes blood collecting and filtering machine for HIV filtration simultaneously detecting a HIV virus, the said device comprising a piston, needle, cameras which causes for push and pull movement of plunger due to this pull push motion of plunger the blood will press toward the tray, which is connected via a filters steel ring. This gives strength to the filter for gentle and smooth filtration of blood for HIV filtration.

The HIV or HIV infected cells or other component of blood is to be passed and thrown across the filter by the pressure of plunger into the blood loaded barrel towards the tray. The filter is fixed between the ends of acrylic cylinder (barrel) of the filtering system to filter the HIV virus or HIV infected cells from the blood filtration and cures the HIV patient. The duration of filtration of HIV or HIV infected cells is dependent with amount of HIV or HIV infected cells present in blood of HIV or aids patient. Suppose a person who is too much infected with HIV virus so the filtration of HIV virus and HIV infected cells can take several days or months, but if a person is infected by HIV in less amounts, so he can cure within few days. Said filtrations are dependent upon how many hours the HIV filtration machine is operated by a patient.

It is an object of the present invention to cure HIV or Aids through the filtration system of blood in which the HIV or HIV infected cells filters directly from the human blood. It will filter the HIV virus from human blood as a treatment of HIV or infected blood for curing HIV or aids patient.

A further important object of the present invention is that, since long back some medicines are available for treatment of aids but these medicine does not cure the aids but such medicine or multi drug can stop to extend of aids for some extend but these medicines are not enough for cure of HIV or aids patient.

A further object of the present invention is that the patient shall call for a blood test. After the report of blood test, it can be decided how much time it will take the HIV filtering machine to filter the HIV from this particular HIV positive patient.

It is another object of the present invention that it is very easy to determine the size of filter as per the requirement it can be changed.

A further very convenient object of the present invention is that, two filtering machines can be put in simultaneously to the patient, for HIV one micron capacity filtering machine for HIV filtration and another machine for the HIV infected T cells filtration, as the filter size of HIV and T cells are different.

It is a further object of the present invention for providing the portable machine which can be put in an arm of HIV or aids patient or it can be fixed on other part of the body of infected person and tightened by a belt. A person can be walking or sitting at any place and the filtration machine can be operated for filtration of HIV virus or HIV infected cells in the said HIV collection tray connected along with the filter's steel ring, because the weight of machine is very less than 250 gr.

The present invention relates to a machine for filtration of infected blood such as HIV virus, T-cells filtration and method of filtration using the machine, and detection of HIV virus in the body using microscopic camera during the process. The said process/machine comprising, an acrylic cylindrical tube (barrel) as shown in FIG. 1 (10) having been divided in to three portions i.e. needle portion FIG. 1 (2), barrel portion (10), and tray FIG. 1 (16) with camera portion, wherein a needle portion containing three needle points i.e. 1^{st} FIG. 1 (3), 2^{nd} FIG. 1 (4), 3^{rd} FIG. 1 (5). These needle points are detachably connected with three blood carrying tubes in the acrylic tube (barrel) which are serially fixed into the three point holes of needles.

Via three blood carrying tubes of the three needle points, the third needle blood carrying tube FIG. 1 (27) is penetrated via head of plunger as shown in FIG. 1 (22) up to the last end of plunger, said 3^{rd} blood carrying tube is dedicated for the purpose of collecting the blood into the acrylic cylinder (barrel) for HIV filtration, and said 3^{rd} tube is closed with a valve FIG. 1 (21) provided, and said blood carrying tube dedicated for storing the blood into acrylic cylinder for HIV filtration purpose. The blood will be taken according the requirements of filtration of HIV or HIV infected WBC or T cells. After collecting the blood in to the cylinder there after HIV filtration can start by pressing a white button which is provided on the body of battery. The 1^{st} and second (FIG. 2) blood carrying tubes joints with 1^{st} (27) and 2^{nd} (28) FIG. 3 point holes of needle and penetrated through the head of plunger up to the last end of plunger both blood carrying tubes are provided with a valve FIG. 1 (12) at the end point of said blood carrying tube. A steel bled is connected with internal space of plunger, connected via battery for power supply, the steel bled which functions as switch for the purpose of opening and closing the valves of 1^{st} and 2^{nd} blood carrying tube, the function of blood carrying tube for throwing and pressing the blood into a vein and cylinder for the purpose of automatic maintain the amount of blood in to the cylinder for the purpose of gently filtering process.

The 1^{st} blood carrying tube FIG. 1 (27) is provided for getting blood from the vein and drops it in to the acrylic cylinder which is used as a barrel with the help of valve at the time of HIV filtration.

The said 2^{nd} blood carrying tube FIG. 1 (28) is used for pulling a drop of blood or more from the barrel via said valve and pressing it into the vein at the time of HIV filtration, the function of the dropping and pressing the blood is used for maintaining the amount of blood in the acrylic cylinder for smooth filtration purpose for HIV, even than a quantity of blood of acrylic cylinder is less than the requirement of filtration. Like one ml or more it can be taken to open the shutter FIG. 1 (6) which provided at 3rd needle point manually and then it would be closed, and filtration process could be started.

The filtering machine can be operated without the two blood carrying tubes along with two valves at the end of said blood carrying tubes, the third blood carrying tube which connected via 3^{rd} needle point the shutter can be opened manually and shut after the required blood is obtained in the acrylic tubes. The HIV machine can be operated without the valve and can drop the blood into the barrel via 3^{rd} blood carrying tube and the shutter of third blood carrying tube can be operated manually.

A piston as shown in FIG. 1 (9,36) detachably fixed above the plunger having small hole on right side of head of the pump, the spring of one psi or more pressure is connected with the above pump, which creates push and pull movement causing push and pull movement of the plunger. Due to this push and pull movement of the plunger, the blood will press towards the tray, which is connected via a filter steel ring (14), the pressure required for pull and pushing capacity of like one pound of pressure spring (the pressure can be increase or decrease by replacing the spring) is connected by a hook on right side head of plunger for the purpose of ups and down movement of plunger along with the ups and down movement of piston, due to the ups and downs movement of piston the plunger does ups and downs movement, in to the blood loaded acrylic cylinder (barrel) due to the pressure of plunger in to the blood loaded acrylic cylinder (barrel) the blood compressed towards the filter so the HIV gets passed across the filter into the tray, hence said HIV filtration process get starts.

The said piston is connected with a circuit of rechargeable battery (8) which supplies power. Then the said piston runs the plunger movement accordingly, said first blood carrying tube which is having a valve at the end point it carries the blood from the vein and throws a drop of blood into the acrylic cylinder (barrel) at the time of upward motion of plunger, said second plastic tube also having a valve at the end point of it, caries the blood from the acrylic cylinder (barrel) and presses a drop of blood into the vein, at the time of downward motion of plunger said valve opens and close due to the movement of steel bled in to the plunger (dropping amount of blood can be increased or decreased to replace the valves).

Said filter FIG. 1 (14) fixed between the ends of the acrylic cylinder, for filtration of dangerous blood components such as HIV virus, is having one inch round shape with the filtering capacity hole of one micron more or less, fixed at the center of half centimeter width steel ring for proving the strength to the filter.

The said filter having one inch diameter steel ring FIG. 5 (48) around the filter the side of steel ring is used for inserting one inch diameter (diameter can change) of acrylic cylinder (barrel) and second side of steel ring is used for inserting one inch diameter of acrylic tray, which is used for collecting the filtered HIV or HIV infected cells or T cells. The filter plays a role of a wall between acrylic cylinder (barrel) and acrylic tray.

The filters are divided into various types such as first filter, second filter, and third filter as shown in FIG. 8, passes the HIV through the filter and throws into the tray for disposal of HIV virus. The filter can be replaced with less than 0.0001 micron to 12 micron or according to the requirement of filtration of HIV or T cells or other components of blood. The blood compress towards the filter via different filter capacities, finally it passes through 0.0001 micron, two filtering machines can be put in simultaneously along with the aids patient, said HIV 0.0001 said filter is having different sectors of different micron membranes so that the all component of blood could be pass like wbc, rbc etc.

The said acrylic tray is one inch diameter in round shape and connected with steel ring of filter and having component of data transmission to the remote computer or mobile device. Said tray has a tiny chip and micro camera FIG. 1 (23) with microscopic lenses.

The said tiny chip and microscopic camera would transmit the data of HIV virus of the different part of human body for information of the presence of HIV into the particular part of human blood, or body and transmit the data signal of particular part of human body's HIV to the remote computer or mobile phone for the help of remote observation. If the said tray is providing a component for data transmission of filtered HIV, T cells or other component of blood to the remote computer, the tray is connected by circuit via rechargeable battery for power therein.

If HIV virus infected cells are detected in any part of body, the virus or HIV infected cells can be observed by using a tiny chip with critical geometrical features smaller than a human hair to observe big volumes of blood in a very gentle and uniform manner to putting a microchip.

The entire human body can be searched by the tiny chip and a micro camera, if HIV viruses or HIV infected cells found in to the any part of human body it can be observed and transmit via transmission data signals via microscopic lenses large image to the computer.

A method an electric power pump of 25 to 90 psi can be used for receiving the blood from catheter and it will be pumped into the body via 8.5 microns filter and this said filter will send the blood for 0.03 micron filter which will filter the virus, and this 0.03 micron filter will pass the blood in to the 0.0001 micron filter for filtering the HIV particle this some amount of other blood components and chemicals will also pass along with HIV particles so again these chemicals will get pass in some less than 0.0001 micron filter, so the maximum amount of blood chemicals will again push toward the catheter tube which will send it to the body via catheter input tube, the two tubes which are connected via these all filters, one for pushing blood into the body and another tube which comes from the filter having size from 6 microns to 0.0001 microns for by passing the HIV viruses, and cd 4 from the blood and it will be collected in to the HIV collecting tray like this the patient will become HIV or aids free. All different filters are used for filtration of blood as shown in FIG. 8.

The blood will come from aids patient via catheter and it will go into the aids patient meanwhile the HIV and HIV infected cells will be filtered due to said microns filters.

### BRIEF DESCRIPTION OF THE DRAWINGS

FIG. 1 is a schematic illustration of the method in the said invention in which including a human hand and said hand's two veins, blood transporting tube, and HIV collection tray.

FIGS. 2-6 illustrate various portions of the device.

FIG. 7. is a schematic illustration of the method said invention for filtering HIV virus from the blood.

FIG. 8. is a schematic illustration of the method in said invention of the three filters of different microns, each filter having two sectors and all three filters connected with each other with an straight pipe, said pipe is fixed in to the center of filters.

FIG. 9. is a schematic illustration of the method in the said invention showing a machine having a display unit, pump, off/on button, and a chamber for wiring and power therein.

FIG. 10 is a further schematic illustration of the method of the invention.

### DETAILED DESCRIPTION OF THE INVENTION

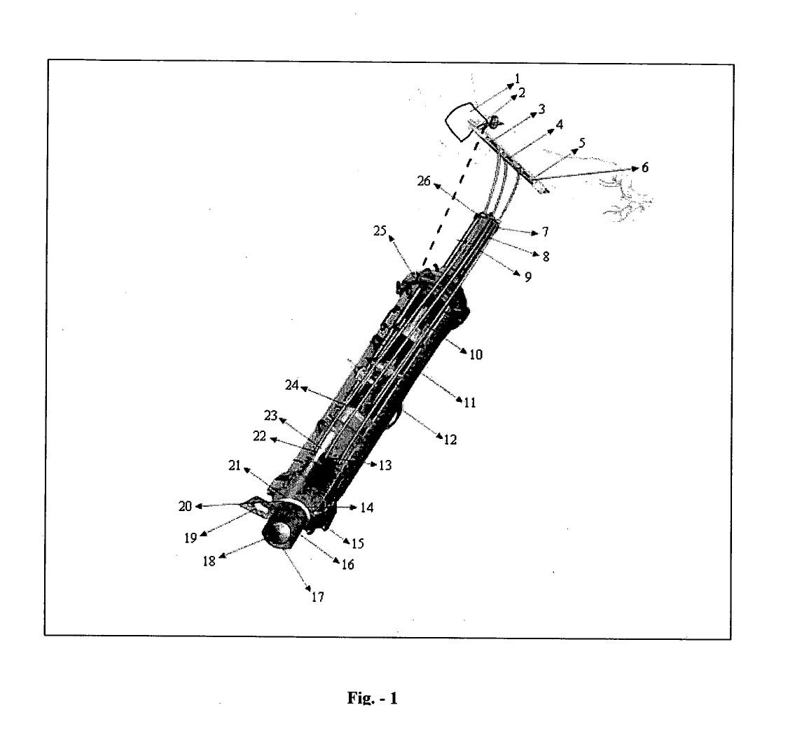

Referring to FIG. 1 the said method for filtering the HIV/Aids from Human blood with a portable HIV Filtration machine includes the following step: FIG. 1,1, a needle injected to the human vein for getting the blood for HIV/HIV Infected cells or CD for filtration purpose. FIG. 1,6 is three holes needed which injected into the vein of HIV or Aids (Acquire immune deficiency syndrome) patient. FIG. 1,3 is a hole on the head of needle, this hole is used for inserting a blood transporting tube for storing the blood in to the barrel for HIV filtration purpose FIG. 1,4 is a second in a serial. This FIG. 1,4 hole is used for inserting a tube for getting one or more drop of blood from the vein. FIG. 1,5 is a another hole in to the serial for getting one drop or more blood from the barrel of these holes 4,5 are used for maintaining the amount of blood at the time of filtration. FIG. 1,6 is a hook of shutter which can be push towards 3 after collecting the blood in the barrel. After requirement of blood into the barrel the hook will be pushed, and it will cover the hole 4. FIG. 1,24 are valves which are on and off with the movement of plunger FIG. 1,24.

FIG. 1,23 is a tube which inserted into the plunger and another tube inserted serially into the head hole FIG. 1,3. FIG. 1,3 used for storing the blood of HIV patients in the barrel for HIV or CD for filtration purpose. Another tube FIG. 1,22 also inserted in the needle hole of FIG. 1,4 this tube carries one or more drops of blood from the vein to the barrel. FIG. 13 is tube which one shown on FIG. 1,22 is inserted through the hole of plunger of the end of plunger which does not shown in the FIG. 1. And said another end of said tube is inserted in to the FIG. 1,4 the FIG. 1,26 is through hole by which the blood transporting tube passes through the cover of battery FIG. 1,9 up to the last end of plunger FIG. 1,23.

Another tube FIG. 1,4 also leads through battery cover 9, in to end of barrel is a cover of the battery. FIG. 1,9 ups due to the plunger connection to the hole of battery cover FIG. 1,9 and FIG. 1,8 is shown the mark of entire battery cover FIG. 1,19 is chip, which controls micro camera and micro chip in the human body via FIG. 1,3.

Between FIG. 1,19 to FIG. 1,3 FIG. 1,25 is a through hole between the entire body of machines, the wire of micro camera & chip to the FIG. 1,2. FIG. 1,10 is a piston connected with the clam of piston which is not shown in the drawing. Said plunger, FIG. 1,24 piston pressure between 20 to 90 PSI becomes up and down due to the movement of said piston FIG. 1,10 said FIG. 1,11 is a illustration of whole body of machine. FIG. 1,12 is a belt, one end of belt connected with body of machine and another end of belt is used for tighten on the arm of an Aids patient said belt's rest of parts is used for tighten on any part of the body. FIG. 1,14 is an inner slot of steel ring FIG. 1,21 of steel ring. FIG. 1,14 is a steel ring which cover the filter of FIG. 1,15 and FIG. 1,15 filter is fixed into the slot of FIG. 1,14 steel ring, filter can connected into the serial which not shown in the FIG. 1,15. FIG. 1,15 can be changed to the 12 microns membranes filter to the 0.0001 micron membrane filter and FIG. 1,15 filter is having different micron membranes into the different sectors filter like 12 microns to 0.0001 for filtering the blood component said components will be remain send into the blood like 12 microns to 0.0001 microns filtering the blood for HIV filtration, like said blood components will be remain send the to the blood and HIV and HIV infected T cells by pass from the blood said tray FIG. 1,16 is connected with slot of FIG. 1,14 steel ring. FIG. 1,18 is radiating tube which will be deactivating the HIV and HIV infected cells into the HIV collecting tray. FIG. 1,19 is a chip and controller which have the connection with FIG. 1,2.

A detailed description of the different components of portable HIV Filtration Machine are separately described.

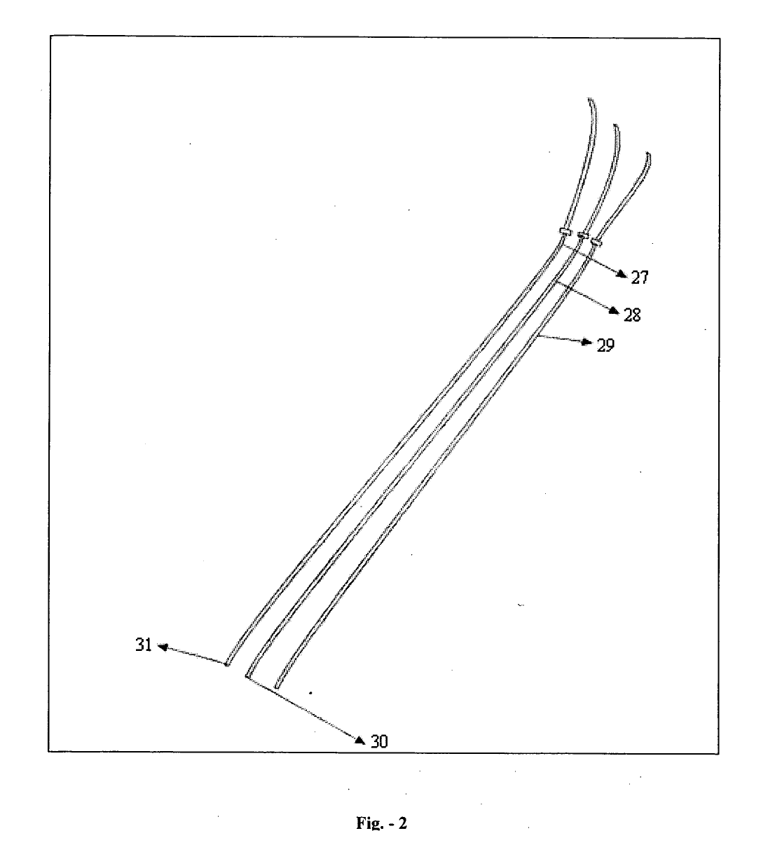

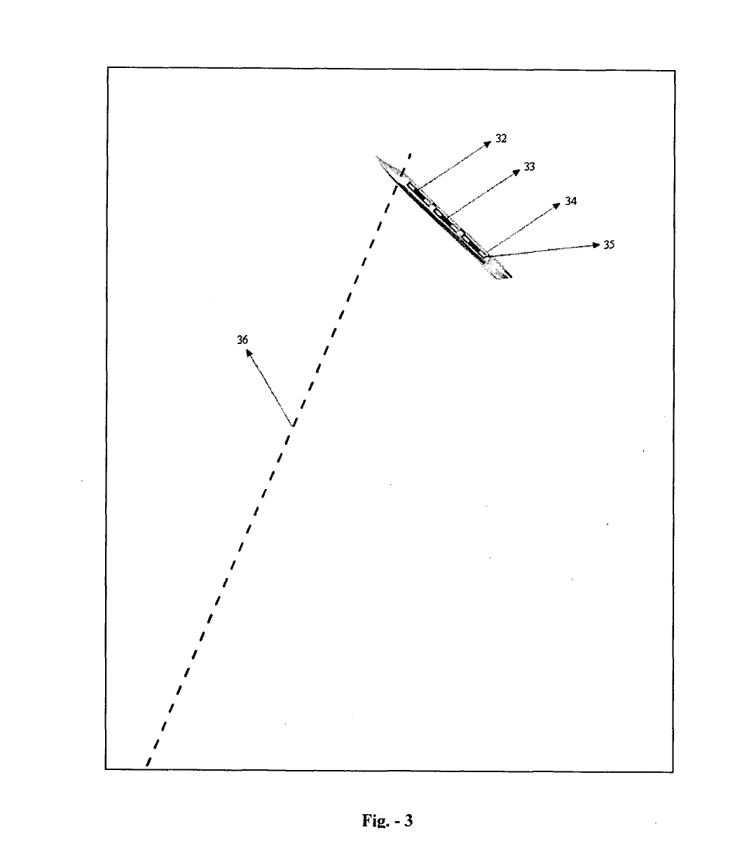

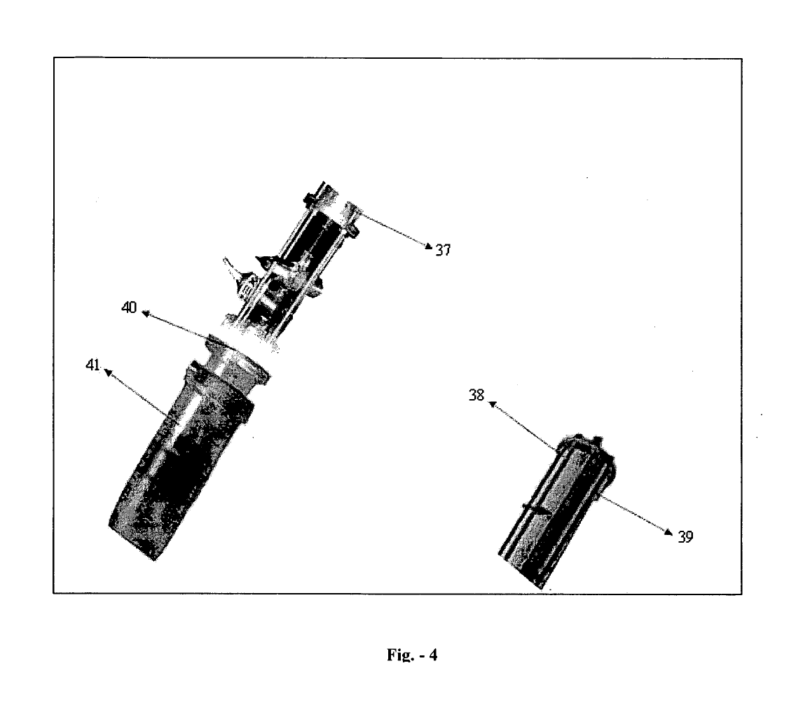

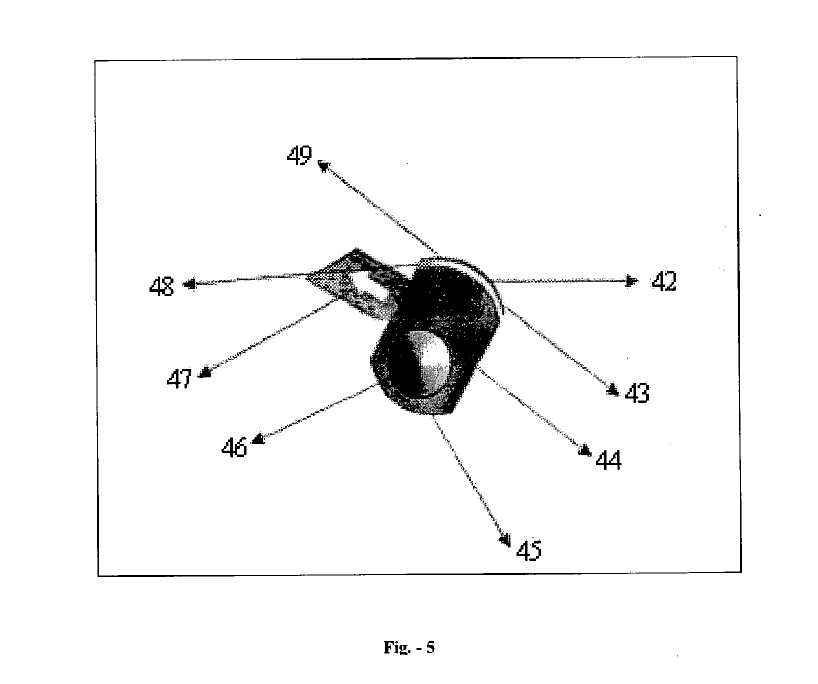

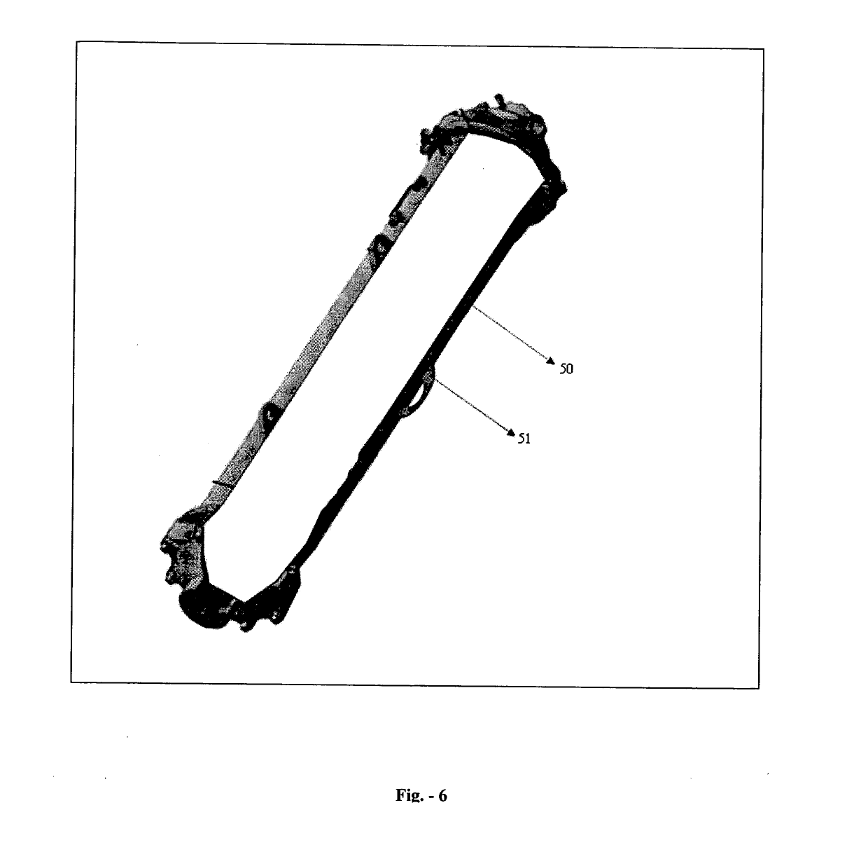

Referring to the FIG. 2,27 is a blood transporting tube also one end is inserted in to the FIG. 1,3 and another end is inserted in to the last point of barrel and last end of FIG. 2,31. FIG. 1,22 said tube FIG. 1,28 is also inserted into the last end of plunger FIG. 1,23. FIG. 2,28 is a tube which carries the blood from the vein is inserted into the FIG. 1,4 show the said tube FIG. 2,28 is connected with the FIG. 1,4. The said tube FIG. 2,28 is connected with the FIG. 1,4. FIG. 2,28 is blood dropping tube which bring the blood from the barrel FIG. 1,13 to the FIG. 1,4 its last end of FIGS. 2,30,—and 29 is a tube which carry one or more drops of blood from the barrel FIG. 1,24 to FIG. 1,5 and last end of said tube is connected via plunger to the last end of the plunger FIG. 1,13 and now illustration of FIG. 3,32 is a hole for blood transporting tube FIG. 3,32; another hole tube of blood transportation from the vein in to the barrel FIG. 3,34 another hole on the head of needle which also into the serial of FIG. 3,32 and FIG. 3,34 is a blood carrying tube which carries the blood, one or more drops from the barrel in to the vein. This makes purpose of required blood for the HIV filtration purpose FIG. 4 is a separate drawing of the component of HIV filtration machine FIG. 4,37 is a piston which moved up and down due to the said piston's movement the plunger gets ups and downs movement the blood compress towards the filter for filtration of HIV/HIV infected cells T cells. FIG. 4,38 is cover of rechargeable battery or power their in FIG. 4,39 is another side of said same battery. FIG. 4,40 is a plunger which is inserted into the barrel and FIG. 4,41 is a barrel which collects the blood for filtration purpose. FIG. 5 is another description of HIV filtration machine FIG. 5,42 is filter of different microns, which can be inserted in to the slot of steel ring FIG. 5,42 is a filter which fixed into the slot of FIG. 5,43. FIG. 5,43 is a steel ring having a slot of round shaped around the steel ring FIG. 5,44 is a HIV/HIV infected cells tray which is fixed in to the back portion of steel ring FIG. 5,42 and FIG. 5,45 is entire body of HIV collection tray. FIG. 4,46 is a camera glass of the HIV filtration tray. FIG. 5,47 is a micro chip of HIV filtration machine. FIG. 4,48 is another part of the said steel ring. FIG. 5,49 is another part of said filter. FIG. 6 is a complete drawing of the body of HIV filtration machine. FIG. 6,50 is shown as a whole body of HIV filtration machine in which all components of HIV filtration are installed and FIG. 6,51 is a belt for tightening the portable HIV filtration machine.

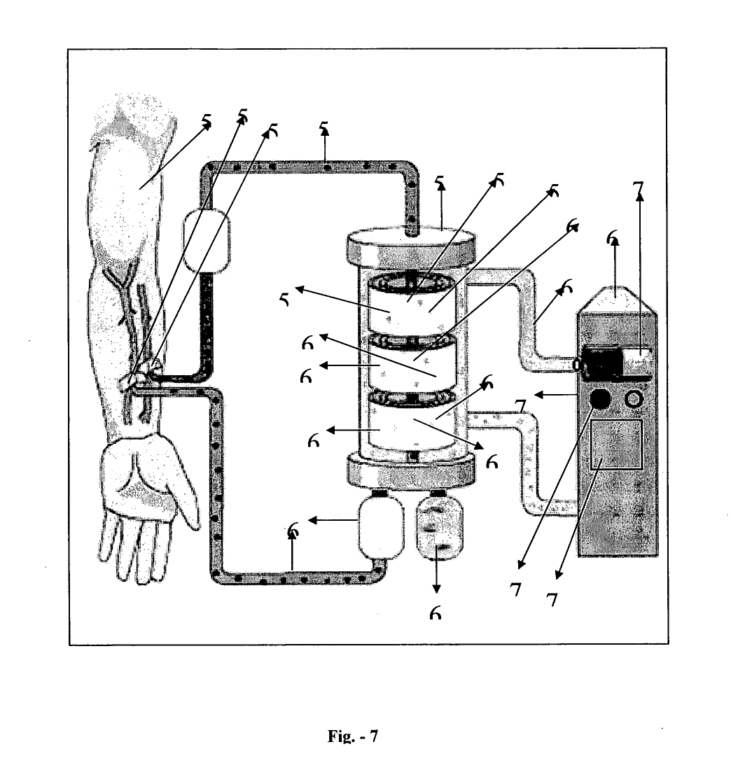

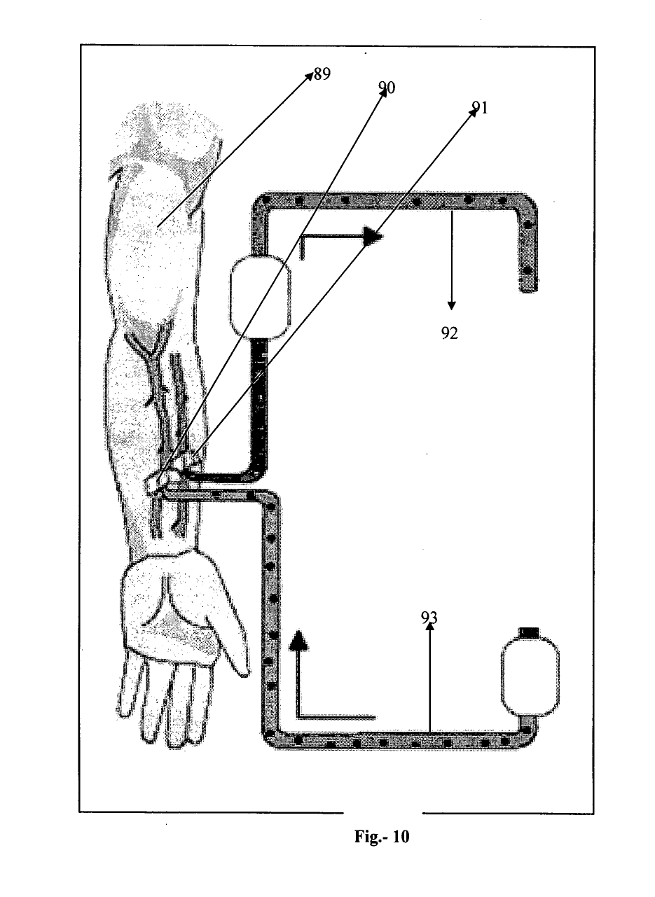

Description of another drawing of HIV filtration machine non pharmacotherapy, said machine is also having the same purpose of HIV filtration is a method of filtration into the different method. Shape of said machine and principles of said machine are different from the machine which start from FIG. 1,1 to FIG. 6,51 and said drawing starts from FIG. 7,52 to FIG. 10,93.

FIG. 7. is a schematic illustration of the method said invention for filtering HIV virus from the blood.

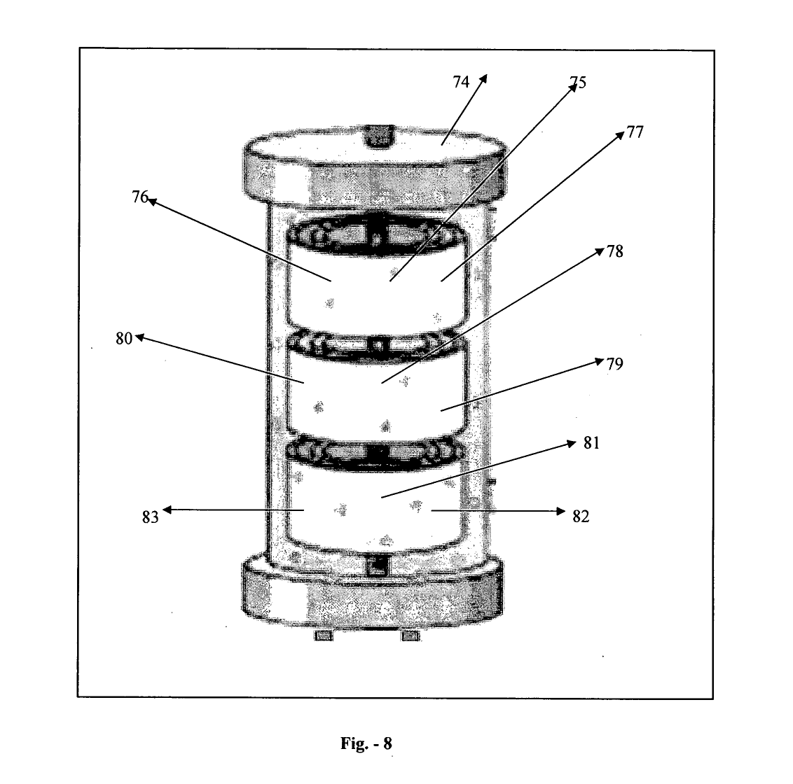

FIG. 8. is a schematic illustration of the method in said invention of the three filters of different microns, each filter having two sectors and all three filters connected with each other with said straight pipe, said pipe is fixed into the center of filters.

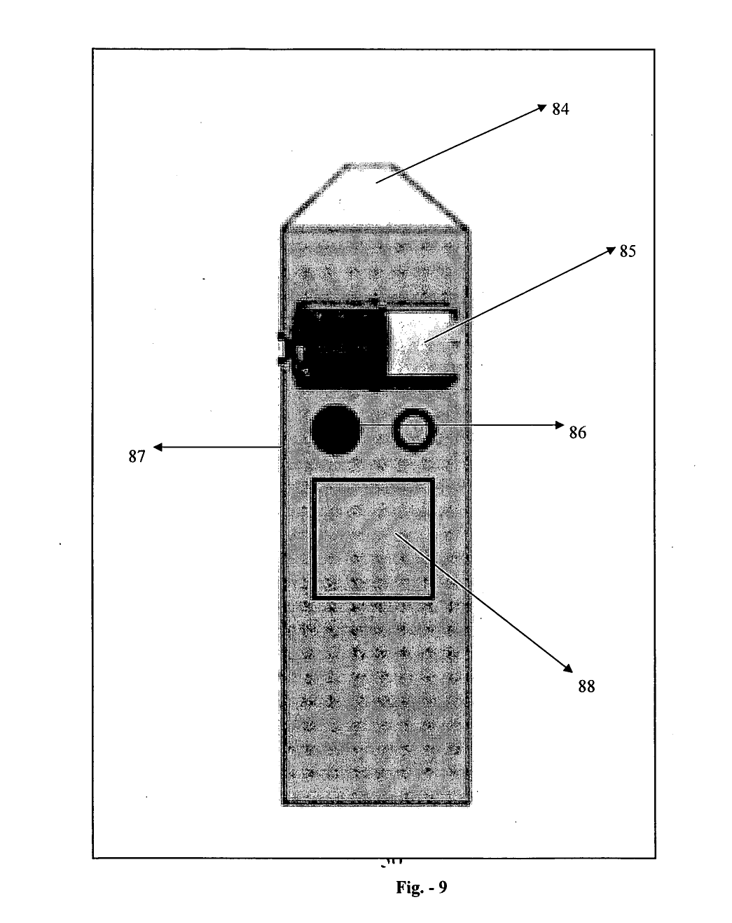

FIG. 9. is a schematic illustration of the method, in the said invention is a machine in which a display unit, pump, off/on button, and a chamber for wiring and power is included therein.

FIG. 1 is a schematic illustration of the method in the said invention in which a human hand and said hand's two vein, blood transporting tube, and HIV collection tray are included.

Referring to FIG. 7,52 is a human hand, FIG. 7,53 and FIG. 7,54 are the veins via said vein FIG. 7,53 and FIG. 7,54 FIG. 7,53 is used for sucking the blood from the vein by the said pump of 20 to 90 PSI and FIG. 7,54 is a vein where again the blood pumps after the HIV and HIV infected cells filtration, HIV or T cells by pass from the blood due to the different microns filters said microns size is between 12 microns to 0.0001 microns. FIG. 7,55 is a tube which transports the blood from the vein and sends it for the filtration with the help of pump. FIG. 7,57 is a filter having two sectors of different microns. FIG. 7,58 is the left sector of the said filter which is having the size of 12 microns. FIG. 7,59 is another sector of same said filter which is having the size of 6 microns FIG. 7,61 is another sector of said filter which fixed with first one. FIG. 7,56 filter also having has two sectors. FIG. 7,61 is a sector of 5 microns and FIG. 7,60 is a section of said filter of 0.01 microns and FIG. 7,65 is a third filter which is also fixed with second filter said 3rd filter is also having two sections. FIG. 7,64 is a section of 5 microns membranes and FIG. 7,65 is a section of 0.0001 microns membranes filter said all filters can be change when required. FIG. 7,66 is a 300 ML capacity HIV bag, FIG. 7,67 is a curve tube which pumps the blood in to the vein. FIG. 7,68 is a tube which is connected with FIG. 7,55 internal tube which is not shown in to the figure. FIG. 7,69 is a display unit of the said machine. FIG. 7,70 is a pump said pump is used for pumping the blood towards the filter for HIV filtration machine. FIG. 7,70 is a figure of whole said machine FIG. 7,72 is an on/off switch for operating the said machine. FIG. 7,73 is a frame for wiring switching and power therein.

FIG. 8,74 is a head of three combined filters. FIG. 8,75 is a first filter FIG. 8,76 is a sector of the said filter FIG. 8,77 is an other sector of filter FIG. 8,78 is a second filter FIG. 8,79 is a second portion of said filter.

What is claimed is:

1. A compact HIV filtration device, comprising: an acrylic cylindrical tube divided into three portions i) needle portion, ii) barrel portion, and iii) tray with camera portion, wherein the needle portion has three needle points, said needle points are detachably connected with three blood carrying tubes serially fixed to the barrel portion, through a head of a plunger up to a last end of plunger, a valve provided at the end point of said blood carrying tubes, a piston detachably fixed above the plunger having a small hole on the head of a pump, connected with a spring of required pressure for creating push and pull movement of the plunger towards the tray through a filter, wherein said tray is one inch or less diameter in round shape and connected with steel ring of filter fixed between the end of the acrylic cylindrical tube, and having connected with a chip and a micro camera, for data transmission to a remote computer or mobile device, which is connected via the filter having a fiber cylindrical half inch diameter tube having an inlet (c) for collecting blood and an outlet (d) for recirculation of unfiltered blood fitted at the bottom of the housing (f), the sizes of the inlet having one centimeter and outlet having a size of the one fourth of the inlet is provided for entry of blood in to the tube, on the tube are six pores (e) having one and half inch size made for passing the blood through the pores, the inlet (c) is detachably attached on the upper and lower side of the housing (f) with a cap (g) of the housing, the outlet of the filter is attached with fixed bottom hole of housing; at the steel ring, said filter having less than 1 micron to more than 8.5 micron pore sizes.

2. The HIV filtration device according to claim 1, wherein the filter is divided into six sectors having pore size varying from 0.0001 to 8.5 microns, wherein the first sector is 0.001 micron, second sector 0.1 to 0.9 micron attached with the first sector, the third sector 1 micron attached with the second sector, the fourth sector having pore sizes 1 to 2 microns attached with the third sector, the fifth sector having pore sizes 2 to 5 microns attached with the fourth sector, and finally the sixth sector having 3 to 8.5 microns attached with the fifth sector by attaching means to make an improved filter.

3. The HIV filtration device according to claim 1, wherein the filter is made up of polypropylene material having different pore sizes starting from 0.0001 to 8.5 microns, constructed by stretching of membrane up to 0.01 micron to 0.09 micron (a) further a 1 micron polypropylene membrane is taken and stretched to create a pore size from 1 to 8.5 microns used as a second sector membrane (b).

4. The HIV filtration device according to claim 1, wherein the filter is a single filtration membrane having six sectors made into a single sheet, having different pore sizes in length or width, the length and width of the membrane can be changed.

5. The HIV filtration device according to claim 4, wherein the length of the filtration membrane is 24 inches and the width being 8.5 inches.

6. The HIV filtration device according to claim 1, wherein the inlet has a 1 centimeter hole and the outlet being one fourth centimeter of the inlet due to variation in the pore sizes of inlet and outlet, by which blood could be accumulated in the tube, due to which and continuous inlet of blood automatically pressure will be developed by which a pressure developed and the accumulated blood could pass through the holes of the tube via the pores of the filter, unfiltered blood will pass through outlet of the tube will again sent for refiltration, and filtered residue could be collated through the outlet of the housing will fall into the collecting tray.

7. The HIV filtration device according to claim 1, wherein the 3^{rd} blood carrying tube is dedicated for the purpose of collecting the blood into the acrylic cylindrical tube for HIV filtration, and said 3^{rd} tube is closed with a valve provided.

8. The HIV filtration device according to claim 1, wherein the 1^{st} blood carrying tube is provided for getting blood from a vein and drops it in to the acrylic cylindrical tube which is used as a barrel with the help of valve at the time of HIV filtration.

9. The HIV filtration device according to claim 1, wherein the 2^{nd} blood carrying tube provided for pulling a drop of blood from the acrylic cylindrical tube via said valve and press it in to a vein at the time of HIV filtration which maintains the amount of blood in to the acrylic cylindrical tube.

10. The HIV filtration device according to claim 1, wherein a shutter is provided at the 3rd needle point that can be operated manually, after obtaining a required quantity of blood.

11. The HIV filtration device according to claim 1, wherein a start button is provided on a body of a battery to start the device.

12. The HIV filtration device according to claim 1, wherein the piston is connected with a circuit of a rechargeable battery which supplies power to run the piston and starts the plunger movement.

13. The HIV filtration device according to claim 1, wherein a steel bled is provided within internal space of the plunger, connected via battery for power supply, the said steel bled which functions as a switch for the purpose of opening and closing the valves of 1^{st} and 2^{nd} blood carrying tubes, the function of blood carrying tube for throwing and, pressing the blood into a vein and cylinder for the purpose of automatically maintaining the amount of blood into the cylinder.

14. The HIV filtration device according to claim 1, wherein a half centimeter steel ring is provided around the filter for inserting one inch or less diameter of the acrylic cylindrical tube and second side of steel ring is used for inserting one inch or less diameter of acrylic tray, which is used for collecting the filtered HIV or HIV infected cells or T cells.

15. The HIV filtration device according to claim 13, wherein said first blood carrying tube having a valve at the end point of it carries the blood from a vein and throws a drop of blood into the acrylic cylindrical tube at the time of upward motion of plunger, said second plastic tube too having a valve at the end point, caries the blood from the acrylic cylindrical tube and press a drop of blood into the vein, during downward motion of plunger said valve opens and closes due to the movement of steel bled into the plunger.

16. The HIV filtration device according to claim 1, wherein the said tiny chip and microscopic camera transmit the HIV presence data of human body and signal of particular part of human body's to the remote computer or mobile phone for observation and for further process.

17. The HIV filtration device according to claim 1, wherein the electric power pump of 25 to 90 psi is used for receiving the blood and can be pumped into a body via filter and send the blood for 0.03 micron filter which will filter a virus, and this 0.03 micron filter will pass the blood into the 0.0001 micron filter for filtering the HIV particle and other blood components and chemicals.

18. A method of HIV filtration, comprising the steps of:
obtaining a device comprising an acrylic cylindrical tube divided in to three portions i) needle portion, ii) barrel portion, and iii) tray with camera portion, wherein the needle portion has three needle points, said needle points are detachably connected with three blood carrying tubes serially fixed to the barrel portion, through a head of a plunger up to a last end of plunger, a valve provided at the end point of said blood carrying tubes, a piston detachably fixed above the plunger having a small hole on the head of a pump, connected with a spring of required pressure for creating push and pull movement of the plunger towards the tray through a filter, wherein said tray is one inch or less diameter in round shape and connected with steel ring of filter fixed between the end of the acrylic cylindrical tube, and connected with a chip and a micro camera, for data transmission to a remote computer or mobile device, which is connected via the filter having a fiber cylindrical half inch diameter tube having an inlet (c) for collecting blood and an outlet (d) for recirculation of unfiltered blood fitted at the bottom of the housing (f), the sizes of the inlet having one centimeter and outlet having a size of the one fourth of the inlet is provided for entry of blood in to the tube, on the tube are six pores (e) having one and half inch size made for passing the blood through the pores, the inlet (c) is detachably attached on the upper and lower side of the housing (f) with a cap (g) of the housing, the outlet of the filter is attached with fixed bottom hole of housing; at the steel ring, said filter having less than 1 micron to more than 8.5 micron pore sizes, and
filtering blood with the device.
